(19) United States
(12) Patent Application Publication
(10) Pub. No.: US 2024/0159938 A1
(43) Pub. Date: May 16, 2024

(54) Compositions for Ophthalmologic Devices

(71) Applicant: Johnson & Johnson Vision Care, Inc., Jacksonville, FL (US)

(72) Inventors: Frank Neely, Jacksonville, FL (US); Kevin P. McCabe, St. Augustine, FL (US); Christopher Chad Harrell, St. Johns, FL (US); Emily B. Carr, Oak Harbor, WA (US)

(21) Appl. No.: 18/538,133

(22) Filed: Dec. 13, 2023

Related U.S. Application Data
(63) Continuation-in-part of application No. 17/546,434, filed on Dec. 9, 2021.
(60) Provisional application No. 63/136,370, filed on Jan. 12, 2021.

(51) Int. Cl. G02B 1/04; A61K 31/439; A61K 47/02; A61K 47/18; C08G 77/26

(52) U.S. Cl. CPC G02B 1/043; A61K 31/439; A61K 47/02; A61K 47/183; C08G 77/26

(57) ABSTRACT

The present invention relates to a sealed ophthalmic product or kit, comprising a package comprising a sealed compartment comprising at least one sterile silicone hydrogel contact lens and a buffered packaging solution having a low pH.

### RELATED APPLICATIONS

The present application is a continuation in part of U.S. patent application Ser. No. 17/546,434, filed Dec. 9, 2021, which claims the benefit of U.S. provisional patent application 63/136,370, filed Jan. 12, 2021, the entirety of which application is hereby incorporated by reference herein as if fully set forth herein.

### FIELD OF TECHNOLOGY

The present invention further relates to low pH buffered packaging solutions for silicone hydrogel contact lenses. Methods of using the compositions of the present invention are also disclosed. Methods of using the compositions of the present invention are also disclosed.

### BACKGROUND OF THE INVENTION

Contact lenses are generally provided to consumers as individually packaged products. The single unit containers which package such contact lenses typically use neutral pH buffered saline as storage or packaging solutions.

Such packaging solutions should provide for, at least in some cases, a short-term period—e.g., between solution preparation and sterilization of the end-staged packaged product—an environment that does not facilitate the growth of harmful or undesirable microorganisms. Moreover, the packaging solutions should be gentle to the eye since at least some of the packaging solution will, most likely, remain on a contact lens once it is removed from the packaging solution and placed directly on (i.e., by direct application to) the eye.

The contact lens (or other ophthalmic device) packaging solution should also be compatible with the materials forming the contact lens (or other ophthalmic device). It is known the presence of acids. For example, U.S. Pat. No. 8,470,906 discloses that the modulus of PureVision® contact lenses can increase from 155 psi to 576 psi when heated at 95° C. for one week due to the hydrolysis of the tris[trimethylsiloxy]silane groups in the presence of small amounts of the acidic comonomer Vinal. The patent suggests limiting the concentration of acidic monomeric in the presence of hydrolytically unstable silicones below a certain threshold.

U.S. Pat. No. 8,921,449 discloses that sterically hindered silicone monomers are more hydrolysis resistant that silicones containing tris[trimethylsiloxy]silane groups, like TRIS. However, such sterically hindered silicone monomers are not widely commercially available and may be expensive to make. Silicone hydrogel lenses are also often preferred by eye care professionals and patients for their balance of properties, including oxygen permeabilities above about 60, 70 or 80 barrers.

A challenge in preparing packaging solutions for ophthalmic devices is formulating solutions which do not negatively affect eye comfort or the solution's compatibility with the material(s) forming the ophthalmic device, including the pH. Contact lenses have also been used as carriers for soluble ophthalmic therapeutic agents. However, not all therapeutic agents are stable at neutral pH (about 7 to about 7.5). Thus, there remains a need for contact lens packaging solutions which can be used with silicone hydrogel lenses at pH values below neutral pH.

### DESCRIPTION OF FIGURES

FIG. 1 is a graph showing microbial colony forming units (CFU) of Staphylococcus aureus present at Day 0, 1 and 2 of challenge with certain compositions of the present invention.

FIG. 2 is a graph showing microbial colony forming units (CFU) of Pseudomonas aeuroginosa present at Day 0, 1 and 2 of challenge with certain compositions of the present invention.

FIG. 3 is a graph showing microbial colony forming units (CFU) of Escherichia coli present at Day 0, 1 and 2 of challenge with certain compositions of the present invention.

25 FIG. 4 is a graph showing microbial colony forming units (CFU) of Bacillus subtilis present at Day 0, 1 and 2 of challenge with certain compositions of the present invention.

FIG. 5 is a graph showing microbial colony forming units (CFU) of Salmonella typhimurium present at Day 0, 1 and 2 of challenge with certain compositions of the present invention.

FIG. 6 is a graph showing microbial colony forming units (CFU) of Candida albicans present at Day 0, 1 and 2 of challenge with certain compositions of the present invention.

FIG. 7 is a graph showing microbial colony forming units (CFU) of Aspergillus brasiliensis present at Day 0, 1 and 2 of challenge with certain compositions of the present invention.

FIG. 8 is a graph showing microbial colony forming units (CFU) of Fusarium keratoplasticum present at Day 0, 1 and 2 of challenge with certain compositions of the present invention.

FIG. 9 is a graph (Chart 9) below presents a comparison of microorganism reduction percentage on each EXP buffer vs the Control Buffer.

FIG. 10 is a graph showing P. aeruginosa CFU/mL Recovery Counts (1350 CFU/mL Day 0 Delivery Count in PBS) at Day 1, 2 and 3 of challenge with a certain composition of the present invention.

FIG. 11 is a graph showing P. aeruginosa Log reduction at Day 1, 2 and 3 of challenge with a certain composition of the present invention.

FIG. 12 is a graph showing percent count reduction of P. aeruginosa at Day 3 of challenge with a certain composition of the present invention.

### SUMMARY OF THE INVENTION

The present invention relates to a sealed ophthalmic product or kit, comprising a contact lens package comprising a sealed compartment comprising at least one sterile silicone hydrogel contact lens and a buffered packaging solution having a pH of about 5 to 6.

The present invention further relates to a sealed ophthalmic product comprising a contact lens package comprising a sealed compartment comprising at least one sterile silicone hydrogel contact lens and a buffered packaging solution having a pH of about 4.5 to 6.5, and osmolality of about 240 to about 600 mOsm/kg, and at least one non-ionic tonicity agent in an amount between about 0.5 wt% and amounts that increase the osmolality of the packaging solution by up to about 280 mOsm/kg.

The present invention further relates to a sealed ophthalmic product comprising a package comprising a sealed compartment comprising at least one sterile ocular insert and a buffered packaging solution having a pH of about 4.5 to 6.5.

The present invention also relates to methods of making and using the disclosed compositions.

### DETAILED DESCRIPTION OF THE INVENTION

As indicated above, the present invention relates to compositions comprising a borate compound and a phosphate compound as an ophthalmologically acceptable carrier.

The present invention further relates to low pH packaging solutions that can be used with silicone hydrogel contact lenses. The inventors have surprisingly found that silicone hydrogel lenses are stable in such solutions despite their known hydrolytic instability when even small concentrations of acidic monomers are included in their formulations.

The compositions may be useful for storing or as a packaging solution for ophthalmic devices, specifically soft contact lenses, and particularly silicone hydrogel contact lenses.

The compositions and methods of the present invention can comprise, consist of, or consist essentially of the steps, essential elements and limitations of the invention described herein, as well any of the additional or optional ingredients, components, or limitations described herein. The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

Unless otherwise indicated, all documents cited are, in relevant part, incorporated herein by reference; the citation of any document is not to be construed as an admission that it is prior art with response to the present invention. Furthermore, all documents incorporated herein by reference are only incorporated herein to the extent that they are not inconsistent with this specification.

The present invention as disclosed herein may be practiced in the absence of any compound or element (or group of compounds or elements) which is not specifically disclosed herein.

As used herein, “pharmaceutically acceptable” means biologically tolerable, and otherwise biologically suitable for application or exposure to the eyes and surrounding tissues of the eyes without undue adverse effects such as toxicity, incompatibility, instability, irritation, allergic response and the like.

As used herein, “hydrolysable group” means a group or moiety which is convertible to hydrogen by hydrolysis or solvolysis. Hydrolyzable groups can be hydrolyzed (i.e., converted to a hydrogen group) by exposure to water or a protic solvent at temperatures and pressures inherent in the packaging, sterilizing and storing of contact lenses, including elevated pressure and temperatures used during autoclaving of contact lenses.

As used herein, “lower alkyl” means a C₁ or C₂ alkyl group.

As used herein, “hydrolyzable silicone monomer” means silicone monomers comprising at least on lower alkyl substituted silanoxyl group, and particularly one, two or three methyl substituted silanoxyl groups, such as 3-[tris(trimethylsiloxy)silyl] group.

All percentages, parts and ratios are based upon the total weight of the composition of the present invention, unless otherwise specified. All such weights as they pertain to the listed ingredients are based on the active level and, therefore, do not include carriers or by-products that may be included in commercially available materials, unless otherwise specified.

### Buffers

A variety of buffers may be used in the present invention, including one or more phosphate compound, sulfate compound, borate compound or organic acid buffers. The buffer may be a phosphate buffer system comprising one or more phosphate compound. The buffer may be a borate buffer system comprising one or more borate compounds. The buffer may comprise an organic acid buffer comprising one or more organic acid buffer or a mixture of organic acid compounds and borate or phosphate compounds. The buffer has the buffer capacity and is present in an amount effective to buffer the composition to a relatively low pH (below neutral), including a pH of from about 4.5 to a pH of about 6.5, from about 4.5 to about 6.0 and from about 5.0 to about 6.0.

### The Borate Compound

The compositions of the present invention comprise a borate. As used herein, the term “borate” shall refer to boric acid, salts of boric acid and other pharmaceutically acceptable borates, or combinations thereof. Suitable borates include, but are not limited to, boric acid; pharmaceutically acceptable salts, such as alkaline metal salts such as sodium borate, potassium borate; alkaline earth metal salts such as calcium borate, magnesium borate; transition metal salts such as manganese borate; and mixtures thereof.

The borate compound can be present in the compositions at concentrations of from about 0.1 wt %, preferably from about 0.2 wt %, preferably from about 0.25 wt %, to less than or equal to 2 wt % (or about 2 wt %), 1.5wt % (or about 1.5 wt %), 1.1wt % (or about 1.1 wt %), in each case, of the total packaging solution. The borate compound may be present in the compositions at concentrations of from about 0.1% wt % to less than or equal to 2 wt % (or about 2 wt %) , from about 0.2 wt % to less than or equal to 1.5 wt % (or about 1.5 wt %), or from about 0.25 wt % to less than or equal to 1.1% (or about 1.1 wt %) of the packaging solution.

### The Phosphate Compound

The compositions of the present invention comprise a phosphate compound. As used herein, the term “phosphate” shall refer to phosphoric acid, salts of phosphoric acid and other pharmaceutically acceptable phosphates, or combinations thereof. Suitable phosphates may be incorporated as one or more monobasic phosphates, dibasic phosphates and the like. Examples of phosphate compounds useful in the compositions are those selected from pharmaceutically acceptable phosphate salts of alkali and/or alkaline earth metals. The phosphate compound may include one or more of sodium dibasic phosphate (Na₂HPO₄), sodium monobasic phosphate (NaH₂PO₄), and potassium monobasic phosphate (KH₂PO₄).

The phosphate compound can be present in the compositions at concentrations of from 0.3 wt % (or about 0.3 wt %) to 0.9 wt % (or about 0.9 wt %), from 0.4 wt % (or about 0.4 wt %) to 0.85 wt % (or about 0.85 wt %), from 0.5 wt % (or about 0.5 wt %) to 0.8 wt % (or about 0.8 wt %) or from 0.6 wt % (or about 0.6 wt %) to 0.75 wt % (or about 0.75 wt %) w/v of the total packaging solution.

When a mixture of at least one phosphate and at least one borate compound are used, the concentration of the phosphate compound may be at least 1.5 (or about 1.5), preferably at least 2.0 (or about 2.0), and preferably at least 2.5 (or about 2.5), but up to 4, preferably up to 3, times the amount of the borate compound on a weight basis.

The ratio of the phosphate compound to the borate compound may be from 1.5:1 (or about 1.5:1) to 3:1 (or about 3:1) preferably from 2:1 (or about 2:1) to 3:1 (or about 3:1) or preferably 2:1 (or about 2:1) on a weight basis.

### The Organic Acid Buffer

The compositions of the present invention comprise an organic acid buffer wherein the organic acid buffer is added to compositions of the present invention in its undissociated form or as the metal salt thereof. As used herein, the term “organic acid buffer” means an organic acid having two or more carboxylic acid groups.

Preferred organic acid buffers for use in the compositions of the present invention have a or pK₂ value in the range of 6 (or about 6) to 8 (or about 8), preferably 6 (or about 6) to 7 (or about 7).

Suitable diprotic acids include maleic acid (pK₂=6.5). Suitable hexaprotic acids include mellitic acid (pK₆=7). Also useful herein is phytic acid (or salts thereof such as their potassium or sodium salts). Phytic acid has 12 replaceable protons, whereby six are strongly acidic (pKa approximately 1.5), three are weaker acidic (pKa between 5.7 and 7.6), and three are very weakly acidic (pKa>10.0) (Costello, A. J. R.; Glonek, T.; Myers, T. C., 1976: ³¹P-nuclear magnetic resonance-pH titrations of myo-inositol hexaphosphate. Carbohydrate Research 46, 159-171). Mixtures of the above acids may also be used.

The organic acid buffer may be selected from phytic acid, mellitic acid, maleic acid and salts thereof (such the sodium or potassium salts of the organic acids) and mixtures thereof. The organic acid buffer may be selected from maleic acid, its sodium or potassium salts and mixtures thereof. The organic acid buffer may be selected from mellitic acid, its sodium or potassium salts and mixtures thereof.

The organic acid content (as sum of mono- and di-basic salts) of the present compositions is in the range of about 0.10% to about 0.4%, or about 0.18% to about 0.30%, or about 0.20% to about 0.28%, by weight the total weight of the composition.

The organic acid buffer may be a combination of salts of the dibasic organic acid anion (e.g., dibasic sodium maleate monohydrate) and salts of the monobasic organic acid anion (monobasic sodium maleate) where the concentration of the dibasic organic acid anion is from about 0.1% to about 0.3% and the concentration of the monobasic organic acid anion is from 0.005% to about 0.002%, by weight of the composition, when present as the metal (e.g., sodium) monohydrate in the case of the dibasic organic acid.

### An Ophthalmologically Acceptable Carrier

The compositions of the present invention comprise an ophthalmologically acceptable carrier. The ophthalmologically acceptable carrier may be water or an aqueous excipient solution. The term “aqueous” typically denotes a formulation wherein the excipient is at least about 50%, more preferably at least about 75% and in particular at least about 90% and up to about 95% or preferably about 99%, by weight, water. The water is distilled water. The carrier may be free of C₁₋₄ alcohols such as methanol, ethanol, propanol, isopropanol, butanol, and the like which can sting, irritate, or otherwise cause discomfort to the eye.

The water may be present in the ophthalmologically acceptable carrier at concentrations of from about 96% to about 99.9%, preferably, from about 98% to about 99.5%, or preferably, from about 99.0% to about 99.5% by weight of the total composition.

The ophthalmologically acceptable carrier may be present at concentrations of from about 96% to about 99.5%, preferably, from about 98% to about 99.5%, or preferably, from about 98.5% to about 99.2% by weight of the total composition.

The compositions may be sterile, namely such that the absence of microbial contaminants in the product prior to release or use are statistically demonstrated to the degree necessary for such products. The compositions may be selected to have no or substantially no detrimental, negative, harmful effect on the contact lens being therein or on the eye (or on the region around the eye).

The compositions according to the present invention are physiologically compatible with the eye and ophthalmic devices. Specifically, the composition should be “ophthalmically safe” for use with an ophthalmic device such as a contact lens, meaning that a contact lens treated with the solution is generally suitable and safe for direct placement on or direct application to the eye without rinsing, that is, the solution is safe and comfortable for ophthalmic devices, of any frequency of application, wetted with the solution, including contact lenses of any wear frequency. An ophthalmologically safe composition has a tonicity and pH that is compatible with the eye and includes materials, and amounts thereof, that are ophthalmically compatible and non-cytotoxic according to ISO standards and U.S. Food & Drug Administration (FDA) regulations.

The compositions of the present invention may be adjusted with tonicity agents, to approximate the osmotic pressure of normal lacrimal fluids, which is equivalent to a 0.9 percent solution of sodium chloride or 2.5 percent of glycerol solution. The compositions may be made substantially isotonic with physiological saline used alone or in combination with other tonicity agents such as glycerol, otherwise if simply blended with sterile water and made hypotonic or made hypertonic the ophthalmic devices such as contact lenses may lose their desirable optical parameters. Correspondingly, excess saline may result in the formation of a hypertonic composition, which will cause stinging, and eye irritation. The osmolality of the composition may be at least about 200 mOsm/kg, preferably from about 200 to about 450 mOsm/kg, preferably from about 205 to about 380 mOsm/kg, preferably from about 210 to about 360 milliosmoles per kilogram (mOsm/kg), preferably from about 250 to about 350 mOsm/kg, or, preferably, from about 300 to about 330 mOsm/kg. The ophthalmic compositions will generally be formulated as sterile aqueous compositions. Both ionic salts and non-ionic compounds like propylene glycol and polyethylene glycols (for example, PEG 400) may be used as tonicity agents to adjust the osmolality. The ionic salts may be present in the composition of the present invention in concentrations to provide osmolalities of at least about 240, about 260 about 280 or about 300 mOsm/kg. The ionic salts may be included in concentrations to provide an osmolality from the salts alone of about 240 mOsm/kg to about 450 mOsm/kg, about 260 mOsm/kg to about 430 mOsm/kg, about 280 mOsm/kg to about 420 mOsm/kg or 260 mOsm/kg to about 300 mOsm/kg. When both ionic salts and non-ionic compounds are present the osmolality of the composition may be about 240 to about 600, about 260 to about 500, about 280 to about 500, about 300 to about 500 or about 200 to about 430 mOsm/kg. Osmolality may be measured by USP<785>.

Examples of suitable tonicity adjusting agents include, but are not limited to, glycerin, sodium, potassium, calcium, zinc and magnesium chloride, alkali metal halides, dextrose, propylene glycol, polyethylene glycols and the like and mixtures thereof. These agents may be used individually in amounts ranging from about 0.01 to about 2.5% w/v and preferably from about 0.2 to about 1.5% w/v. When propylene glycol or polyethylene glycol or mixtures thereof are used as non-ionic tonicity agents they may be included in amounts that increase the osmolality of the packaging solution by up to about 150, 200 or 280 mOsm/kg. The propylene glycol, may be included in amounts between 0.5 to about 3 wt %, 0.5 to about 2 wt % or about 0.5 to about 1.5 wt %. The polyethylene glycol may be included in amounts between about 0.5 to about 3 wt %, or about 0.5 to about 2 wt %, or about 0.5 to about 1.5 wt %. A suitable polyethylene glycol is polyethylene glycol 400.

The tonicity adjusting agent may be sodium chloride which can be incorporated at concentrations of from about 0.4 to about 0.9, preferably, from about 0.4 to about 0.7, or preferably, from about 0.5% to about 0.6% by weight of the total composition.

The compositions of the present invention may have a pH of from about 4.5 to about 6.5, 5.0 to a pH of about 6.0.

The pH of the ophthalmic composition may be adjusted using acids and bases, such as mineral acids, such as, but not limited to hydrochloric acid and bases such as sodium hydroxide.

The compositions of the present invention are useful as packaging solutions for packaging of ophthalmic devices.

As used herein, “ophthalmic device” refers to an object that resides in or on the eye. These devices can provide optical correction or may be cosmetic. Ophthalmic devices include but are not limited to soft contact lenses, intraocular lenses, overlay lenses, ocular inserts, punctual plugs, and optical inserts. The ophthalmic device may be an ocular inserts which can be placed in or on the ocular environment, such as between the eye and eyelid. Ocular inserts may be with silicone materials, such as mono-(2-hydroxy-3-methacryloxypropyloxy)-propyl terminated mono-n-butyl terminated polydimethylsiloxanes (OH-mPDMS) (containing from 4 to 30, or from 4 to 20, or from 4 to 15 SiO repeat units).

The ophthalmic device may be a contact lens. Contact lenses useful with the compositions can be manufactured employing various conventional techniques, to yield a shaped article having the desired posterior and anterior lens surfaces. Spincasting methods are disclosed in U.S. Pat. Nos. 3,408,429 and 3,660,545; static casting methods are disclosed in U.S. Pat. Nos.4,113,224, 4,197,266, and 5,271,875, each of which are herein incorporated by reference. Contact lens polymer materials useful for manufacturing suitable contact lenses include, but are not limited to, acofilcon A, alofilcon A, alphafilcon A, amifilcon A, astifilcon A, atalafilcon A, balafilcon A, bisfilcon A, bufilcon A, comfilcon, crofilcon A, cyclofilcon A, darfilcon A, deltafilcon A, delefilcon, deltafilcon B, dimefilcon A, drooxifilcon A, epsifilcon A, esterifilcon A, etafilcon A, fanfilcon A, focofilcon A, galyfilcon A, genfilcon A, govafilcon A, hefilcon A, hefilcon B, hefilcon D, hilafilcon A, hilafilcon B, hioxifilcon B, hioxifilcon C, hixoifilcon A, hydrofilcon A, lenefilcon A, licryfilcon A, licryfilcon B, lidofilcon A, lidofilcon B, lotrafilcon A, lotrafilcon B, mafilcon A, mesifilcon A, methafilcon B, mipafilcon A, narafilcon A, narafilcon B, nelfilcon A, netrafilcon A, ocufilcon A, ocufilcon B, ocufilcon C, ocufilcon D, ocufilcon E, ofilcon A, omafilcon A, oxyfilcon A, pentafilcon A, perfilcon A, pevafilcon A, phemfilcon A, polymacon, riofilcon A, samfilcon A, senofilcon A, senofilcon C, silafilcon A, siloxyfilcon A, somofilcon A, stenfilcon A, tefilcon A, tetrafilcon A, trifilcon A, vasurfilcon, vifilcon, and xylofilcon A. Prefereably, the contact lenses are manufactured using polymer materials selected from (or selected from the group consisting of) comfilcon, etafilcon A, galyfilcon A, senofilcon A, nelfilcon A, hilafilcon, tetrafilcon A, vasurfilcon, vifilcon, and polymacon.

Silicon hydrogel formulations include balafilcon samfilcon, lotrafilcon A and B, delfilcon, galyfilcon, senofilcon A, B and C, narafilcon, comfilcon, formofilcon, riofilcon, fanfilcon, stenfilcon, somofilcon, kalifilcon and the like. “Silicone hydrogels” refer to polymeric networks made from at least one hydrophilic component and at least one silicone-containing component. Silicone hydrogels may have moduli in the range of 60-200, 60-150 or 80 -130 psi, water contents in the range of 20 to 60%. Examples of silicone hydrogels include acquafilcon, asmofilcon, balafilcon, comfilcon, delefilcon, enfilcon, fanfilcon, formofilcon, galyfilcon, lotrafilcon, narafilcon, riofilcon, samfilcon, senofilcon, somofilcon, and stenfilcon, including all of their variants, as well as silicone hydrogels as prepared in U.S. Pat. Nos. 4,659,782, 4,659,783, 5,244,981, 5,314,960, 5,331,067, 5,371,147, 5,998,498, 6,087,415, 5,760,100, 5,776,999, 5,789,461, 5,849,811, 5,965,631, 6,367,929, 6,822,016, 6,867,245, 6,943,203, 7,247,692, 7,249,848, 7,553,880, 7,666,921, 7,786,185, 7,956,131, 8,022,158, 8,273,802, 8,399,538, 8,470,906, 8,450,387, 8,487,058, 8,507,577, 8,637,621, 8,703,891, 8,937,110, 8,937,111, 8,940,812, 9,056,878, 9,057,821, 9,125,808, 9,140,825, 9156,934, 9,170,349, 9,244,196, 9,244,197, 9,260,544, 9,297,928, 9,297,929 as well as WO 03/22321, WO 2008/061992, and US 2010/0048847. These patents are hereby incorporated by reference in their entireties.

The inventors have found that silicone hydrogel contact lenses may be packaged in the low pH packaging solutions of the present invention without degrading the contact lenses even after exposure to elevated temperatures. The silicone hydrogel contact lenses display consistent modulii when packaged and sterilized in low pH packaging solutions (between about 4.5 and about 6.5, about 5 to about 6 or about 5.5), even when the silicone hydrogel comprises monomer units comprising trimethylsilyl groups (—Si(CH₃)₃) (“TMS groups”). It is known in the art that silicone monomers containing TMS groups and contact lenses formed from them are hydrolyzable in the presence of even small amounts of acidic comomoners. “[I]f a carboxylic acid such as methacrylic acid is used as a copolymerization component in order to obtain a higher moisture content, the silicone component is gradually hydrolyzed, so that the physical properties of the contact lens may be degraded when the contact lens is stored for a long time.” U.S. Pat. No. 8,921,449, column 1, lines 25-29.

Examples of TMS-containing silicone monomers include 3-[tris(trimethylsiloxy)silyl]propyl methacrylate (TRIS), 3-methacryloxypropylbis(trimethylsiloxy)methylsilane, N-[3-tris(trimethylsiloxy)silyl]-propyl acrylamide (TRIS-Am), 2-hydroxy-3-[3-methyl-3,3-di(trimethylsiloxy)silylpropoxy]-propyl methacrylate (SiMAA), 2-hydroxy-3-methacryloxypropyloxypropyl-tris(trimethylsiloxy)silane and

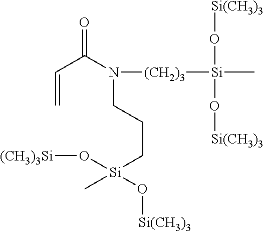

Examples of silicone hydrogel polymers comprising units from hydrolysable silicone monomers include acquafilcon, galyfilcon, senofilcon, stenfilcon, samfilcon, balafilcon, delefilcon. Silicone hydrogel contact lenses comprising monomer units from hydrolysable silicone monomers may be packaged in packaging solutions having pH ranges between about 5 and 6 display stable mechanical properties, including modulii with less than 10% or less than 5% increase even after 1, 2, 3, 4, 5, 6, 7, 8, 9 or 10 autoclave cycles.

The compositions may also be useful for direct application to eye as a wetting or rewetting eye drop for providing relief to eye discomfort (e.g., burning sensations relating to the eye or general eye irritation).

The compositions described herein may be free of or substantially free of preservatives. The term “preservative” means compounds having antimicrobial properties. Examples of specific preservatives include, but are not limited to, 4-chlorocresol, 4-chloroxylenol, benzalkonium, benzalkonium chloride, benzoic acid, benzyl alcohol, chlorhexidine, chlorobutanol, imidurea, m-cresol, methylparaben, phenols 0.5%, phenoxyethanol, sorbate, propionic acid, propylparaben, sodium benzoate, sorbic acid, thimerosol, Stabilized Oxychloro Complex (SOC—99.5% chlorite, 0.5% chlorate, and with trace amounts of chlorine dioxide), polyquaternium compounds (such as polyquarternium-42 polyquarternium-1), perborate salts (e.g., sodium perborate, biguanide compounds (e.g., polyhexamethylene biguanide or polyaminopropyl biguanide).

The term “substantially free” as related to preservatives means that the preservative is present in the compositions of the present invention at a concentration of less than 2% (or about 2%), preferably less than 1.5% (or about 1.5%), and preferably less than 1% (or about 1%), preferably less than 0.5% (or about 0.5%), preferably less than 0.1% (or about 0.1%), preferably less than 0.05% (or about 0.05%), preferably less than 0.01% (or about 0.01%), preferably less than 0.005% (or about 0.005%) by weight of the total composition. Preferably, the compositions of the present invention are free of preservatives.

As mentioned above, contact lenses is immersed in a composition of the present invention and stored in a suitable packaging container, preferably, a packaging container for single contact lens unit. Generally, a packaging container for the storage of a contact lens includes at least a sealing layer sealing the container containing an unused contact lens immersed in the composition of the present invention. The sealed container may be hermetically sealed packaging container. The hermetically sealed packaging container may be a blister pack in which a concave well containing a contact lens is covered by a metal or plastic sheet adapted for peeling in order to open the blister-pack. The sealed container may be formed from any suitable, generally inert packaging material providing a reasonable degree of protection to the lens. The packaging material may be formed of plastic material such as polyalkylene, PVC, polyamide, glass, glassy polymers and the like.

Any water soluble, demulcent (or demulcent like—e.g., having demulcent properties such as viscosity increasing capabilities) polymer may also be employed in the composition of this invention provided that it has no (or no substantial) detrimental effect on the contact lens being stored or on the wearer of the contact lens at the concentrations used in the composition of the present invention or on the eye (or on the region around the eye). Particularly useful components are those, which are water soluble, for example, soluble at the concentrations used in the presently useful liquid aqueous media. Suitable water soluble demulcent polymers include, but are not limited to, demulcent polymers, such as block copolymers of polyethyleneoxide (PEO) and polypropyleneoxide (PPO); polyvinyl alcohol, polyvinyl pyrrolidone; polyacrylic acid; polyethers such as polyethylene glycols (e.g., polyethylene glycol 300, polyethylene glycol 400) and polyethylene oxides; hyaluronic acid, and hyaluronic acid derivatives; chitosan; polysorbates such as polysorbate 80, polysorbate 60 and polysorbate 40); dextrans such as dextran 70; cellulosic derivatives such as carboxy methyl cellulose methylcellulose, hydroxyethylcellulose, hydroxypropylcellulose, and methyl ethyl cellulose; acyclic polyamides such as those having a weight average molecular weight of 2,500 to 1,800,000 Daltons as disclosed in U.S. Pat. No. 7,786,185 herein incorporated by reference in its entirety; salts of any of the above and mixtures of any of the above. Preferably, the block copolymers of PEO and PPO include poloxamers and poloxamines, including those disclosed in U.S. Pat. No. 6,440,366, herein incorporated by reference in its entirety. Preferably, the water-soluble demulcent polymer is selected from polyvinyl pyrrolidone, methyl ethyl cellulose, polyvinyl alcohol, polymethacrylic acid, carboxymethyl cellulose, glycerol, propylene glycol, 1,3-propanediol, polyethylene glycols, and mixtures thereof.

Lubricating agents may have molecular weights in excess of 100,000. When glycerol, propylene glycol and 1,3-propanediol are used as lubricating agents, they may have molecular weights lower than 100,000.

When any water soluble polymer is used in the packing solutions of the present invention, it may be included and present in amounts up to about 0.5, 1 or 2 weight % preferably between about 0.001 and about 2%, between about 0.005 and about 1%, between about 0.01 and about 0.5 weight %, or between about 100 ppm and about 0.5 weight %, all based upon the weight of total composition.

When any water soluble polymer is used in the direct application eye care formulation or eye drop of the present invention, it may be included and present in amounts up to about 2, 5 or 10 weight %, preferably between about 0.001 and about 10%, between about 0.005 and about 2% , between about 0.01 and about 0.5 weight %, or between about 100 ppm and about 2 weight %, all based upon the weight of total composition.

Without being limited by theory, it is believed that the water soluble demulcent polymer aids in preventing the ophthalmic device from sticking to its product packaging and may enhance the initial (and/or extended) comfort of the contact lens, packaged in the composition, when placed on the eye after removal from the packaging.

The demulcent polymer may be a cellulosic derivative. The cellulosic derivative may be present at concentrations of from about 0.002 to about 0.01, or preferably, from about 0.004 to about 0.006 by weight of the total composition of the present invention.

Various other materials may be included with the compositions described herein.

In the case of compositions of the present invention for direct application to the eye, surfactants may be included. Surfactants suitable for such use include, but are not limited to, ionic and nonionic surfactants (though nonionic surfactants are preferred), RLM 100, POE 20 cetylstearyl ethers such as Procol® CS20, poloxamers such as Pluronic® F68, and block copolymers such as poly(oxyethylene)-poly(oxybutylene) compounds set forth in U.S. Patent Application Publication No. 2008/0138310 entitled “Use of PEO-PBO Block Copolymers in Ophthalmic Compositions” filed Dec. 10, 2007 (which publication is herein incorporated by reference)

Surfactant may be present at concentrations of from about 0.01 to about 3%, preferably from about 0.01 to about 1%, preferably, from about 0.02 to about 0.5%, or preferably, from about 0.02 to about 0.1% by weight of the total composition of the present invention.

If desired, one or more additional components may be, optionally, included in the composition. Such optional component(s) are chosen to impart or provide at least one beneficial or desired property to the composition. Such additional, but optional, components may be selected from components that are conventionally used in ophthalmic device care compositions. Examples of such optional components include (or, are selected from or selected from the group consisting of), wetting agents, therapeutic agent, sequestering agents, viscosity builders, antioxidants, and the like and mixtures thereof. These optional components may each be included in the compositions in an amount effective to impart or provide the beneficial or desired property to the compositions such the beneficial or desired property is noticeable to the user. For example, such optional components may be included in the compositions in amounts similar to the amounts of such components used in other eye or ophthalmic device care compositions products.

All components in the ophthalmic solution of the present invention should be water-soluble. As used herein, water soluble means that the components, either alone or in combination with other components, do not form precipitates or gel particles visible to the human eye at the concentrations selected and across the temperatures and pH regimes common for manufacturing, sterilizing and storing the ophthalmic solution.

One or more therapeutic agent may also be incorporated into the ophthalmic solution. A wide variety of therapeutic agents may be used. Suitable therapeutic agents include those that treat or target any part of the ocular environment, including the anterior and posterior sections of the eye and include pharmaceutical agents, vitamins, nutraceuticals combinations thereof and the like. Suitable classes of active agents include antihistamines, antibiotics, glaucoma medication, carbonic anhydrase inhibitors, anti-viral agents, anti-inflammatory agents, non-steroid anti-inflammatory drugs, antifungal drugs, anesthetic agents, miotics, mydriatics, immunosuppressive agents, antiparasitic drugs, anti-protozoal drugs, combinations thereof and the like. When therapeutic agents are included, they are included in an amount sufficient to produce the desired therapeutic result (a “therapeutically effective amount”).

Some ophthalmic therapeutic agents are unstable at neutral pH. It has been surprisingly found that these low pH therapeutic agents may be packaged with a contact lens and a low pH packaging solution of the present invention. Examples of low pH therapeutic agents include antimuscarinic agents, prostaglandins, cholinergic agonists, steroids, antibiotics, anti-inflammatories and dry eye drugs.

Examples of antimuscarinic agents include atropine, atropine sulfate, pirenzepine, racanisodamine, cylcontolate, homatropine, scopolamine, telenzepine, nuvenzepine and rispenzepine. The antimuscarinic agent may be atropine, atropine sulfate or mixtures thereof.

Examples of prostaglandins includine but are not limited to latanoprost, travoprost, bimatoprost, tafluprost and latanoprostene bunod. The prostaglandin may be latanoprost or bimatoprost.

Examples of cholinergic agonists include, but not limited to pilocarpine, acetylcholine, methacholine, carbachol, bethanechol, muscarine and cevimeline. The prostaglandin may be pilocarpine.

Examples of ophthalmic steroids include, but are not limited to hydrocortisone, loteprednol, prednisolone, prednisolone acetate, and dexamethasone.

Examples of antibiotics include, but are not limited to ciprofloxacin, amoxicillin, tobramycin, meomycin, bacitracin, polymyxin B and gentamycin.

Examples of antihistimines and mast cell stabilizers include, but are not limited to ketotifen, ketotifen fumarate, olopatadine, pheniramine, naphazoline, alcaftadine, bepotastine, epinastine, azelastine, phenylephrine, cetirizine, nedocromil and pemirolast. The antihistimines and mast cell stabilizers may be ketotifen, ketotifen fumarate, olopatadine, alcaftadine, azelastine or cetirizine.

Examples of alpha adrenergic agonists include but are not limited to brimonidine, brimonidine tartrate.

Examples of dry eye drugs and anti-inflammatory drugs, include but are not limited to lifitigrast, perfluorohexyloxtane, bromfenac, nepafenac, ketorolac, diclofenac, fluriprofen and suprofen.

The therapeutic agent may be selected from atropine, atropine sulfate, brimonidine, brimonidine tartrate, lifitigrast, perfluorohexyloxtane, ketotifen, ketotifen fumarate, olopatadine, alcaftadine, azelastine, cetirizine, latanoprost, bimatoprost, pilocarpine, hydrocortisone, loteprednol, prednisolone, prednisolone acetate, dexamethasone, ciprofloxacin, amoxicillin, tobramycin, meomycin, bacitracin, polymyxin B and gentamycin. The therapeutic agent may be selected from atropine, atropine sulfate, brimonidine, brimonidine tartrate, lifitigrast, perfluorohexyloxtane, ketotifen, ketotifen fumarate, olopatadine, alcaftadine, azelastine, cetirizine, latanoprost, bimatoprost, pilocarpine, prednisolone, prednisolone acetate and dexamethasone.

The therapeutic agent may be atropine or atropine sulfate. The atropine may be used with a contact lens of any design, including those designed to slow the progression of myopia, such as those disclosed in U.S. Pat. Nos. 6,752,499, 7,832,859, 11,789,292, 11,768,386, 11,754,859, 10,901,237, 8,684,520, 9,977,257, 10,877,294, 9,625,739, 11,822,153, 11,567,347, 11,234,862, 11,467,424.

When an atropine compound is used as the therapeutic agent, the concentration may be about 0.001 to about 1.5 wt %, based upon the total weight of the packaging solution. When used to treat mydriasis suitable concentrations include between about 0.1 to about 1.5 wt %. When used for to slow the progression of myopia, the atropine compound may be present in amounts between about 0.001 to about 0.05 wt %, or about 0.01 to about 0.05 wt % atropine compound based upon the total weight of the packaging solution.

When an atropine compound is used as the therapeutic agent the buffer is selected from organic acid buffers and phosphate compounds and mixtures thereof or is selected from phosphate compounds. In one embodiment, when a therapeutic agent is incorporated in the composition of the present invention with a silicone hydrogel contact lens the buffer is free of borate compounds.

Useful optional sequestering agents include, but are not limited to, disodium ethylene diamine tetraacetate (EDTA), diethylenetriamine pentaacetic acid (DTPA) and ophthalmically compatible salts thereof, alkali metal hexametaphosphate, citric acid, sodium citrate and the like and mixtures thereof. The chelant may comprise EDTA. When present, the chelating agent may be present in amounts from about 0.008 to about 0.075 or about 0.01 to about 0.075 wt %. The chelating agent may also be present in amounts between about 0.008 and about 0.012 wt % or about 0.1 wt %, based on the total packaging solution composition.

Useful optional antioxidants include, but are not limited to, sodium metabisulfite, sodium thiosulfate, N-acetylcysteine, butylated hydroxyanisole, butylated hydroxytoluene and the like and mixtures thereof.

The method of packaging and storing a contact lens (or other ophthalmic device) includes at least incorporating the device into packaging where the device is immersed in the compositions described above. The method may include immersing the device in the composition prior to delivery to the customer/wearer, directly following manufacture of the contact lens. Alternately, the incorporation and storing of the device in the compositions (all in the packaging) may occur at an intermediate point before delivery to the ultimate customer (wearer) but following manufacture and transportation of the device in a dry state, wherein the dry device is hydrated by immersing the device in the compositions. Consequently, a package for delivery to a customer may comprise a sealed container containing one or more unused devices (e.g., contact lenses) immersed in the compositions.

In one preferred embodiment, the steps for packaging the device in the composition of the present invention include:
(1) molding a device (e.g., contact lens) in a mold comprising at least a first and second mold portion,
(2) removing the device from the mold portions and removal of unreacted monomer and processing agents
(3) introducing the composition and the device into the packaging (or container), and
(4) sealing the packaging.

The method also includes the step of sterilizing the contents of the packaging. Sterilization may take place prior to, or most conveniently after, sealing of the container and may be performed by any suitable method known in the art, e.g., by balanced autoclaving of the sealed container at temperatures of about 120° C. or higher. The packaging may be a plastic blister packaging (or package), including a recess for receiving a device and the composition, where the recess is sealed with lidstock prior to sterilization of the package contents. The term “lidstock” as used herein means the foil laminate composite material, including the aluminum foil and the other layers of polymers, that is heat sealed to cover the concave side of the blister.

The following test methods were used in the examples.

Water content was measured gravimetrically. Lenses were equilibrated in packing solution for 24 hours. Each of three test lenses is removed from packing solution using a sponge tipped swab and placed on blotting wipes which have been dampened with packing solution. Both sides of the lens are contacted with the wipe. Using tweezers, the test lens is placed in a tared weighing pan and weighed. Then two more sets of samples are prepared and weighed. All weight measurements were done in triplicate, and the average of those values used in the calculations. The wet weight is defined as the combined weight of the pan and wet lenses minus the weight of the weighing pan alone.

The dry weight was measured by placing the sample pans in a vacuum oven which has been preheated to 60° C. for 30 minutes. Vacuum was applied until the pressure reaches at least 1 inch of Hg is attained; lower pressures are allowed. The vacuum valve and pump are turned off and the lenses are dried for at least 12 hours, typically overnight. The purge valve is opened allowing dry air or dry nitrogen gas to enter. The oven is allowed to reach atmospheric pressure. The pans are removed and weighed. The dry weight is defined as the combined weight of the pan and dry lenses minus the weight of the weighing pan alone. The water content of the test lens was calculated as follows: % water content (% WC)=(wet weight−dry weight)/wet weight×100. The average water content and its standard deviation were calculated, and the average value reported as the percent water content of the test lens. Standard deviations are listed in the table in the parentheses.

The mechanical properties of the contact lenses were measured by using a tensile testing machine such as an Instron model 1122 or 5542 equipped with a load cell and pneumatic grip controls. Minus one diopter lens (spherical) is the preferred lens geometry because of its central uniform thickness profile. A dog-bone shaped sample cut from a −1.00 diopter power lens having a 0.522 inch length, 0.276 inch “ear” width and 0.213 inch “neck” width was loaded into the grips and elongated at a constant rate of strain of 2 inches per minute until it breaks. The center thickness of the dog-bone sample was measured using an electronic thickness gauge prior to testing. The initial gauge length of the sample (Lₒ) and sample length at break (L_{f}) were measured. At least five specimens of each composition were measured, and the average values were used to calculate the percent elongation to break: percent elongation=[(L_{f}−Lₒ)/Lₒ]×100.

The tensile modulus (M) was calculated as the slope of the initial linear portion of the stress-strain curve; the units of modulus are pounds per square inch or psi.

The tensile strength (TS) was calculated from the peak load and the original cross-sectional area: tensile strength=peak load divided by the original cross-sectional area; the units of tensile strength are psi.

Toughness was calculated from the energy to break and the original volume of the sample: toughness=energy to break divided by the original sample volume; the units of toughness are in-lbs/in³.

The elongation to break (ETB) was also recorded as the percent strain at break. Standard deviations of the mechanical properties were calculated and listed in the data tables in parentheses.

The following examples are provided to enable one skilled in the art to practice the compositions and are merely illustrative of the invention. The examples should not be read as limiting the scope of the invention as defined in the claims.

### EXAMPLES

### Example 1

Table 1 shows compositions (i.e., test Exp1-Exp5) useful as solutions for storing or as packaging solution for ophthalmic devices (e.g., contact lenses) together with a control, each of which compositions can be prepared using conventional mixing technology.

TABLE 1

| | Weight % (phosphates corrected for hydrates) | | | | | |
|---|---|---|---|---|---|---|
| Component | EXP1 | EXP2 | EXP3 | EXP4 | EXP5 | Control |
| Deionized | 95.623 | 97.899 | 95.892 | 98.167 | 98.39 | 98.03 |
| Water | | | | | | |
| Sodium | 0.84 | 0.84 | 0.84 | 0.84 | 0.84 | 0.84 |
| Chloride | | | | | | |
| Boric Acid | 0.400 | 0.400 | 0.182 | 0.182 | 0 | 0.91 |
| Sodium | 0.092 | 0.092 | 0.042 | 0.042 | 0 | 0.21 |
| Borate | | | | | | |
| Decahydrate | | | | | | |
| monobasic | 1.047 | 0.262 | 1.047 | 0.262 | 0.262 | 0 |
| sodium | | | | | | |
| phosphate | | | | | | |
| dibasic | 1.987 | 0.497 | 1.987 | 0.497 | 0.497 | 0 |
| sodium | | | | | | |
| phosphate | | | | | | |
| EDTA | 0.010 | 0.010 | 0.010 | 0.010 | 0.010 | 0.01 |

Once prepared, each of the compositions of Table 1 was poured from its original specimen cup containers and filter sterilized through a 0.22 μm membrane using a 150-mL Analytical Filter Unit. The filtered individual compositions were then aseptically transferred into new individual sterile specimen cups for storage and testing.

The following microorganisms were used to assess microbial activity:
Staphylococcus aureus (Quanti-Cult Plus™)—ATCC 6538 (Remel Inc.)
Escherichia coli (BioBall® Multishot 550)—NCTC 12923 (ATCC 8739) (bioMérieux)
Bacillus subtilis (Quanti-Cult Plus™)—ATCC 6633 (Remel Inc.)
Pseudomonas aeuroginosa ((Quanti-Cult Plus™))—ATCC 9027 (Remel Inc.)
Candida albicans ((Quanti-Cult Plus™))—ATCC 10231 (Remel Inc.)
Salmonella typhimurium (KWIK-STIK™)—ATCC 14028 (Microbiologics, Inc.)
Aspergillus brasiliensis ((Quanti-Cult Plus™))—ATCC 16404 (Remel Inc.)
Fusarium keratoplasticum (KWIK-STIK™)—ATCC 36031 (Microbiologics, Inc.)

For the Quanti-Cult Plus and BioBall Multishot microorganisms, a solution 1:1 (microorganism sample to composition) was prepared for each composition of Table 1. An inoculum of 200 μl was used for spread plating in duplicate.

For the remaining KWIK-STIK™ microorganisms (i.e., in swab/pellet presentation), Microbiologics, Inc's instructions were followed for reconstitution. A stock solution was created using the disposable Hemocytometer 2-Chip instructions (Bulldog Bio) for general methods. Based on results obtained, serial dilutions were performed to obtain a countable inoculum for each buffer. Inoculum volume used for spread plating in duplicate was 100 μl.

Bacteria microorganisms were plated in tryptic soy agar (TSA) media and Yeast and Fungi in Sabouraud dextrose agar (SDA) or SDA with chloramphenicol. TSA plates were incubated for 24-72 hrs. at 30-35° C. and SDA plates were incubated for 48 hrs. to 4 days at 20-25° C.

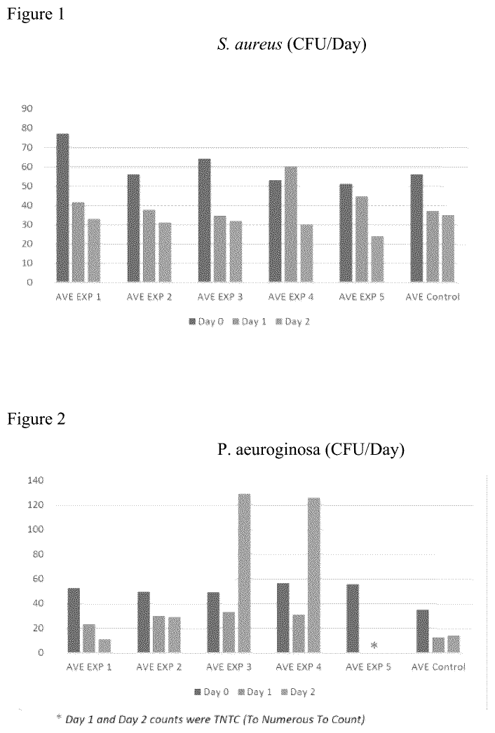

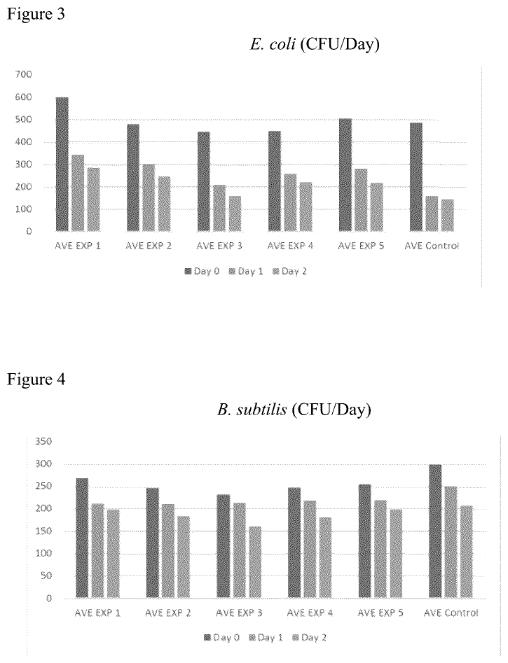

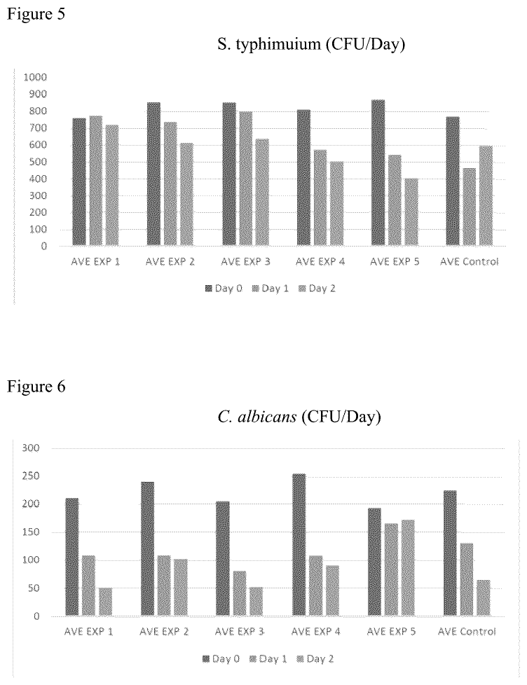

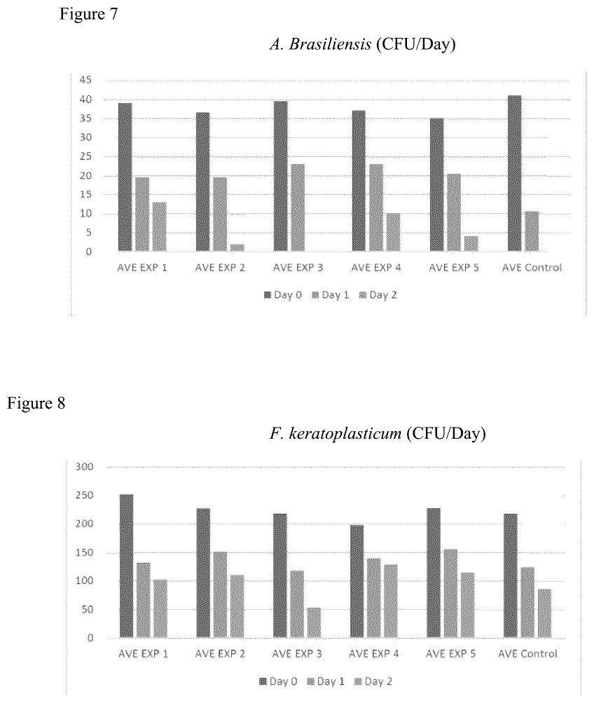

Quantitative analysis was performed on Day 0, 1 and 2. FIGS. 1-8 summarize the results obtained.

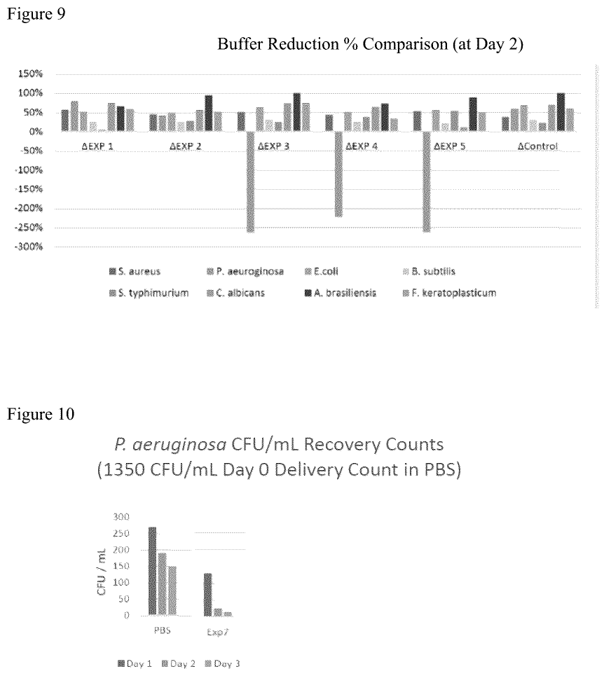

The data in FIGS. 1-8 demonstrate that the compositions of Table 1 provide bacteriostatic/fungistatic capability and bactericidal/fungicidal activity. FIG. 9 below shows a comparison of microorganism reduction percentage for each of compositions EXP1-EXP5 vs the Control after 2 days. Compositions EXP1 and EXP2 show the most equivalent reduction pattern vs the Control and showed stasis (i.e., no increase from the initial count of inoculate microorganism) against all microorganisms after two days. However, composition EXP 2 shows the most consistent reduction % across all challenged microorganisms.

### Example 2

The composition of Table 2 is useful as a solution for storing or as packaging solution for ophthalmic devices (e.g., contact lenses) is prepared as described below using conventional mixing technology.

TABLE 2

| | Weight % (the balance is water) |
|---|---|
| Component | |
| Boric acid | 0.30% |
| Sodium Chloride | 0.55% |
| Monobasic sodium | 0.081% |
| phosphate*H₂O | |
| Dibasic sodium phosphate*7H₂O | 0.67% |
| Disodium EDTA | 0.01% |
| MEC | 0.005% |
| Water | 98.384% |
| Composition Properties | |
| pH | 7.2* |
| Osmolality | 288* |

*Measured in the composition absent the presence of MEC and EDTA-it being recognized that due the concentration of these components, such components alone or in combination would have minor impact on pH and osmolality relative to the remaining components.

Once prepared, the composition of Table 2 was poured from their original specimen cup containers and filter sterilized through a 0.22 μm membrane using a 250-mL Rapid Flow Filtration Unit. The filtered composition was then aseptically transferred into a sterile container for microbial growth testing on Pseudomonas aeuroginosa (Quanti Cult plus)—using Pseudomonas aeuroginosa ATTC culture type no. 9027).

P. aeruginosa (PA) Quanti-Cult plus was resuspended following manufacturers' instructions and an approximately 500 μL aliquot was spread plated onto two separate tryptic soy agar (TSA) plates. The plates were incubated at 30-35° C. for 2 days. An inoculating loop was used to resuspend the PA from the TSA plate surface in OmniPur WFI Quality Water, Sterile Filtered, Calbiochem (WFI).

The suspension was aseptically transferred with a sterile pipette into a 50 mL centrifuge tube.

The PA suspension was serially diluted with WFI quality sterile water and a hemocytometer (Disposable Hemocytometer, Bulldog Bio) was used to obtain a 1:1000 dilution containing a target population count of approximately 1.0×10⁷ cells/mL.

Ten μL aliquot of the 1:1000 PA dilution was inoculated into 40 mL of the composition of Table 1 and a 1×PBS control solution to obtain a starting PA target population count of approximately 2500 CFU/mL. (PBS=AccuGENE 1×Phosphate Buffered Saline, 1.7 mM KH₂PO₄, 5 mM Na₂HPO₄, 150 mM NaCl, pH 7.4, Cat. No. 51225, LONZA). 100 μL of the PBS control solution was plated in triplicate onto TSA plates and incubated at 30-35° C. to determine the Day 0 PA delivery counts.

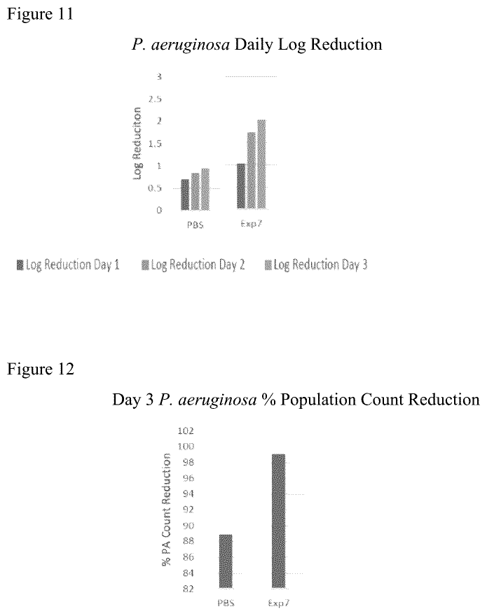

The PA inoculated composition of Table 2 and PBS control were stored at room temperature and at Day 1, Day 2 and Day 3 samples were spread plated (100-300 μL aliquots) in triplicate onto TSA. The plates were then incubated at 30-35° C. to quantitate the PA population counts following room temperature storage. The results for the composition in Table 1 is represented as “Exp. 7” in FIGS. 10-12.

As shown in FIGS. 10-12, the composition of Table 2 (shown as “Exp7”) caused a greater reduction in PA population counts compared to the PBS control and, accordingly, provide the desired bacteriostatic properties. The reduction in PA population provided further evidence of prevention of increase from initial PA population count—or that bacteriostasis was achieved.

### Example 3

The composition of Table 3 is useful as a solution for storing or as packaging solution for ophthalmic devices (e.g., contact lenses) is prepared as described below using conventional mixing technology.

TABLE 3

| | Weight % (the balance is water) |
|---|---|
| Component | |
| Boric acid | 0.29% |
| Sodium Chloride | 0.60% |
| Monobasic sodium | 0.081% |
| phosphate*H₂O | |
| Dibasic sodium phosphate*7H₂O | 0.67% |
| Disodium EDTA | 0.01% |
| MEC | 0.005% |
| Water | 98.394% |
| Composition Properties | |
| pH | 7.2 |
| Osmolarity | 308 |

### Phosphate Buffer (PB) Prep

Phospate buffered contact lens packaging solution (Phosphate Buffer Control) was prepared by adding 2000 g DI water to a 5L vessel. The components listed in Table 4 (other than DI water) were added to the vessel with mixing. The remaining 1937 g water was added and the solution was mixed for 1 hour. All mixing was done at room temperature.

TABLE 4

| Component | Wt % | Supplier |
|---|---|---|
| Sodium chloride | 0.83 | Fischer |
| | | Scientific |
| Sodium Phosphate, Monobasic Monohydrate | 0.1 | Spectrum |
| Dibasic Sodium Phosphate, Heptahydrate | 0.63 | Spectrum |
| ethylene diamine tetraacetic acid (EDTA) | 0.01 | Fischer |
| | | Scientific |
| Methyl ether cellulose | 0.005 | Fisher |
| | | Scientific |
| Deionized water | 98.425 | |

### Borate Buffer (BB) Prep

Borate buffered contact lens packaging solution (BB) was prepared by adding the components listed in Table 5 (other than the methyl ether cellulose) in a non-reactive vessel at room temperature with mixing. The solution was mixed for about 40 minutes. The temperature was raised to about 58° C. and the methyl ether cellulose was added with mixing and mixed for about 13 minutes. The solution was mixed about 80 additional minutes at room temperature.

TABLE 5

| Component | Wt % | Supplier |
|---|---|---|
| Deionized Water | 98.4797 | NA |
| Sodium Chloride | 0.4449 | Sigma-Aldrich |
| Boric Acid | 0.8900 | JT Baker |
| Sodium Borate | 0.1700 | JT Baker |
| Decahydrate | | |
| EDTA | 0.0104 | JJVC-K. Yokus |
| Methyl Ether Cellulose | 0.0050 | Sigma-Aldrich |

### Examples 4-27

ACUVUE Oasys 1Day Brand contact lenses (senofilcon A) were removed from their blister packages, blotted to remove excess original packaging solution and placed into vials (one lens/vial) with 4 ml of the packing solution having the pH and buffer as shown in Table 6 and sealed with a Teflon crimp cap. The pH of the buffers was adjusted using HCl (Sigma Aldrich, ACS Reagent Grade).

Commercial lenses (−1.0) were sterilized once as part of the manufacturing process. Lenses in vials were then subjected to further steam sterilization cycle, 121° C. for 18 min cycle. One cycle being defined as a ramp up in heat to 121° C. then held for 18 min, the ramp down to 40° C. is one cycle.

Lenses were subjected to sterilization cycles of 1, 3, 5, 8 & 10 cycles. The number of sterilizations cycles listed in the Table 6 includes the original manufacturer's sterilization cycle.

A baseline measurement of the lenses was tested as well as after every sterilization cycle for mechanicals & gravimetric water content. Controls were run with BB and PB systems at pH 5.5 and 7.4 for each arm.

TABLE 6

| | | | # | | | | | |
|---|---|---|---|---|---|---|---|---|
| | | | Steri | water | Mod. | | tensile | |
| Ex.# | Buffer | pH | cycles | content | (psi) | ETB | strength | toughness |
| 4 | BB | 5.5 | 0 | 40 (0) | 91 (6) | 231 (35) | 110 (19) | 128 (35) |
| 5 | BB | 5.5 | 1 | 40 (0) | 91 (8) | 237 (28) | 116 (14) | 136 (26) |
| 6 | BB | 5.5 | 3 | 39 (0) | 92 (4) | 224 (35) | 104 (20) | 118 (35) |
| 7 | BB | 5.5 | 5 | 40 (0) | 91 (6) | 213 (44) | 102 (25) | 115 (42) |
| 8 | BB | 5.5 | 8 | 40 (0) | 92 (6) | 216 (25) | 102 (13) | 114 (25) |
| 9 | BB | 5.5 | 10 | 40 (0) | 96 (9) | 178 (38) | 90 (23) | 93 (45) |
| 10 | BB | 7.4 | 0 | 41 (0) | 96 (7) | 204 (41) | 102 (20) | 107 (33) |
| 11 | BB | 7.4 | 1 | 39 (1) | 98 (5) | 184 (47) | 100 (25) | 98 (41) |
| 12 | BB | 7.4 | 3 | 40 (0) | 98 (4) | 192 (52) | 104 (30) | 103 (48) |
| 13 | BB | 7.4 | 5 | 39 (0) | 98 (4) | 245 (27) | 135 (18) | 158 (31) |
| 14 | BB | 7.4 | 8 | 39 (0) | 102 (6) | 208 (32) | 114 (20) | 122 (35) |
| 15 | BB | 7.4 | 10 | 38 (0) | 102 (6) | 203 (46) | 117 (29) | 122 (46) |
| 16 | PB | 5.5 | 0 | 41 (0) | 93 (6) | 238 (21) | 118 (15) | 142 (23) |
| 17 | PB | 5.5 | 1 | 40 (0) | 94 (6) | 221 (48) | 111 (26) | 126 (48) |
| 18 | PB | 5.5 | 3 | 40 (0) | 98 (8) | 204 (34) | 102 (23) | 108 (37) |
| 18 | PB | 5.5 | 5 | 40 (0) | 92 (7) | 209 (50) | 104 (24) | 115 (41) |
| 20 | PB | 5.5 | 8 | 40 (0) | 89 (6) | 198 (35) | 92 (17) | 96 (29) |
| 21 | PB | 5.5 | 10 | 40 (0) | 96 (6) | 203 (38) | 105 (19) | 111 (34) |
| 22 | PB | 7.4 | 0 | 40 (0) | 97 (5) | 217 (33) | 106 (18) | 119 (32) |
| 23 | PB | 7.4 | 1 | 39 (1) | 99 (5) | 201 (37) | 106 (21) | 110 (36) |
| 24 | PB | 7.4 | 3 | 39 (0) | 100 (5) | 206 (28) | 116 (18) | 121 (28) |
| 25 | PB | 7.4 | 5 | 39 (0) | 100 (5) | 196 (30) | 111 (22) | 111 (35) |
| 26 | PB | 7.4 | 8 | 39 (0) | 102 (4) | 194 (35) | 107 (21) | 109 (35) |
| 27 | PB | 7.4 | 10 | 39 (0) | 102 (6) | 213 (47) | 128 (33) | 139 (55) |

The senofilcon A contact lenses showed no mechanical changes even after ten sterilizations in borate and phosphate buffers at a pH 5.5. The stability at low pH was the same as at neutral pH, which is surprising given that including even small amount 1.6 mol % in silicone hydrogels (such as senofilcon A, which includes SIMAA) can cause the modulus to increase at accelerated aging conditions as low as 55° C.

### Examples 28-52

Commercially available contact lenses (-1.0) were removed from their blister packages, blotted to remove excess original packaging solution and placed into vials (one lens/vial) with 4 ml of the packing solution having the pH and buffer as shown in Table 7 and sealed with a Teflon crimp cap.

Lenses in vials were then subjected to steam sterilization, 121° C. for 18 min cycle. One cycle being defined as a ramp up in heat to 121° C. then held for 18 min, the ramp down to 40° C. is one cycle. Lenses were subjected to additional sterilization cycles in the experimental packing solutions. The number of sterilizations cycles listed in the Table 2 includes the original manufacturer's sterilization cycle. A baseline measurement of the lenses was tested (listed as 1 sterilization cycle) as well as after every sterilization cycle for mechanicals & gravimetric water content.

Controls were run with Borate (BB) & phosphate (PB) buffer systems at pH 5.5 & 7.4 for each arm, 4 total.

No discernable changes were observed across all arms and sterilization cycles, suggesting that a variety of silicone hydrogel lenses immersed in a low pH solution may be packaged in packaging solutions having pH values below 6 without adversely affects to the polymer network.

TABLE 7

| | | | | # | | | | | |
|---|---|---|---|---|---|---|---|---|---|
| | | | | Steri | | Mod | | tensile | |
| Ex# | Lens | Buffer | pH | Cycles | [H20] | (psi) | % Elong | strength | Toughness |
| 28 | Oasys | BB | 7.4 | 1 | 40 (0) | 100 (9) | 218 (40) | 124 (23) | 136 (36) |
| | 1Day | | | | | | | | |
| 96 | Oasys | BB | 7.4 | 3 | 40 (0) | 94 (10) | 219 (39) | 110 (19) | 124 (29) |
| | 1Day | | | | | | | | |
| 30 | Oasys | BB | 7.4 | 5 | 40 (0) | 95 (10) | 208 (30) | 113 (22) | 121 (30) |
| | 1Day | | | | | | | | |
| 31 | Oasys | BB | 7.4 | 8 | 40 (0) | 101 (9) | 186 (45) | 97 (23) | 98 (40) |
| | 1Day | | | | | | | | |
| 32 | Oasys | BB | 7.4 | 10 | 41 (0) | 101 (9) | 242 (56) | 141 (47) | 171 (75) |
| | 1Day | | | | | | | | |
| 33 | Oasys | PB | 5.5 | 1 | 40 (0) | 89 (5) | 225 (32) | 96 (15) | 115 (28) |
| | 1Day | | | | | | | | |
| 34 | Oasys | PB | 5.5 | 3 | 40 (0) | 95 (3) | 219 (38) | 92 (18) | 109 (35) |
| | 1Day | | | | | | | | |
| 35 | Oasys | PB | 5.5 | 5 | 40(0) | 97 (4) | 215 (40) | 100 (21) | 116 (40) |
| | 1Day | | | | | | | | |
| 36 | Oasys | PB | 5.5 | 8 | 40(0) | 98 (4) | 197 (36) | 90 (17) | 96 (32) |
| | 1Day | | | | | | | | |
| 37 | Oasys | PB | 5.5 | 10 | 40 (0) | 92 (5) | 200 (27) | 85 (13) | 91 (23) |
| | 1Day | | | | | | | | |
| 38 | MyDay | PB | 5.5 | 1 | 57 (0) | 95 (6) | 256 (35) | 99 (15) | 136 (33) |
| 39 | MyDay | PB | 5.5 | 3 | 58 (0) | 99 (8) | 301 (20) | 125 (11) | 188 (23) |
| 40 | MyDay | PB | 5.5 | 5 | 57 (0) | 91 (4) | 271 (29) | 112 (17) | 158 (35) |
| 41 | MyDay | PB | 5.5 | 8 | 58 (0) | 84 (5) | 265 (58) | 106 (24) | 146 (53) |
| 42 | MyDay | PB | 5.5 | 10 | 57 (0) | 88 (6) | 283 (22) | 117 (12) | 171 (25) |
| 43 | Ultra | PB | 5.5 | 1 | 49 (0) | 101 (8) | 240 (45) | 150 (30) | 183 (63) |
| 44 | Ultra | PB | 5.5 | 3 | 49 (0) | 102 (6) | 245 (59) | 153 (37) | 194 (82) |
| 45 | Ultra | PB | 5.5 | 5 | 49 (0) | 102 (4) | 257 (36) | 167 (23) | 213 (52) |
| 46 | Ultra | PB | 5.5 | 8 | 49 (0) | 106 (4) | 268 (31) | 181 (21) | 238 (48) |
| 47 | Ultra | PB | 5.5 | 10 | 49 (0) | 100 (7) | 259 (25) | 172 (21) | 220 (41) |
| 48 | Biofinity | PB | 5.5 | 1 | 49 (0) | 105 (6) | 240 (52) | 114 (20) | 157 (54) |
| 49 | Biofinity | PB | 5.5 | 3 | 49 (0) | 107 (4) | 240 (42) | 111 (15) | 151 (43) |
| 50 | Biofinity | PB | 5.5 | 5 | 49 (0) | 105 (2) | 243 (55) | 115 (21) | 161 (58) |
| 51 | Biofinity | PB | 5.5 | 8 | 49 (0) | 105 (3) | 202 (54) | 98 (21) | 118 (52) |
| 52 | Biofinity | PB | 5.5 | 10 | 50 (0) | 103 (4) | 247 (33) | 112 (14) | 158 (39) |

Oasys 1Day = ACUVUE ® Oasys ™
1Day Brand Contact Lenses (senofilcon A)
MyDay = MyDay ® Daily Disposible Brand Contact Lenses (stenfilcon)
Ultra- Ultra ® Brand Contact Lenses (samfilcon)
Biofinity = Biofinity ® Brand Contact Lenses (comfilcon)
Standard Deviations are shown in parenthesis

All silicone hydrogel lenses tested showed good stability in mechanical properties even up to ten sterilization cycles. Ultra includes TRIS, which contains three tris[trimethyl(siloxy)] end groups, confirming that a wide range of silicone hydrogel contact lenses are stable at low pH in both borate and phosphate buffer systems with low pH.

Embodiments:
1. A sealed ophthalmic product comprising:
   a contact lens package comprising a sealed compartment comprising at least one sterile silicone hydrogel contact lens and a buffered packaging solution having a pH of about 4.5 to 6.5.
2. The product of any of the preceding or succeeding embodiments wherein the silicone hydrogel contact lens comprises units from at least one hydrolysable silicone monomer.
3. The product of embodiment 2 wherein the hydrolysable silicone monomer is selected from 3-[tris(trimethylsiloxy)silyl]propyl methacrylate (TRIS), 3-methacryloxypropylbis(trimethylsiloxy)methylsilane, N-[3-tris(trimethylsiloxy)silyl]-propyl acrylamide (TRIS-Am), 2-hydroxy-3-[3-methyl-3,3-di(trimethylsiloxy)silylpropoxy]-propyl methacrylate (SiMAA), 2-hydroxy-3-methacryloxypropyloxypropyl-tris(trimethylsiloxy)silane and

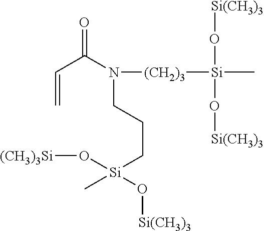

And mixtures thereof.
4. The product of embodiment 2 wherein the hydrolysable silicone monomer is selected from 3-[tris(trimethylsiloxy)silyl]propyl methacrylate (TRIS), 3-methacryloxypropylbis(trimethylsiloxy)methylsilane, N-[3-tris(trimethylsiloxy)silyl]-propyl acrylamide (TRIS-Am), 2-hydroxy-3-[3-methyl-3,3-di(trimethylsiloxy)silylpropoxy]-propyl methacrylate (SiMAA), 2-hydroxy-3-methacryloxypropyloxypropyl-tris(trimethylsiloxy)silane and mixtures thereof.
5. The product of embodiments 1 or 2 wherein the silicone hydrogel is selected from include galyfilcon, senofilcon, stenfilcon, samfilcon, balafilcon, delefilcon.
6. The product of any of the foregoing or preceding embodiments wherein the pH is about 5 to about 6.
7. The product of any of the foregoing or preceding embodiments wherein the buffered packaging solution comprises one or more phosphate compound, sulfate compound, borate compound or organic acid buffers.
8. The product of any of the foregoing or preceding embodiments wherein the buffered packaging solution is a phosphate buffer system comprising one or more phosphate compound.
9. The product of any of the foregoing or preceding embodiments wherein the buffered packaging solution is an organic acid buffer comprising one or more organic acid buffer or a mixture of organic acid compounds and borate or phosphate compounds.
10. The product of any of the foregoing or preceding embodiments wherein the buffered packaging solution is a borate buffer system comprising one or more borate compound.
11. The product of embodiment 10 wherein the composition comprises from about 0.1 wt % to about 2 wt %, from about 0.2 wt % to less about 1.5 wt %, from about 0.25 wt % to about 1.1 wt % of at least one borate compound, by weight of the total weight of the packaging solution.
12. The product of embodiments 7 or 9 wherein the organic acid buffer is selected from phytic acid, mellitic acid, maleic acid and salts thereof (such the sodium or potassium salts of the organic acids) and mixtures thereof.
13. The product of embodiments 7 or 9 wherein the organic acid buffer is selected from maleic acid, its sodium or potassium salts and mixtures thereof.

14. T The product of embodiments 7 or 9 wherein the organic acid buffer is selected from mellitic acid, its sodium or potassium salts and mixtures thereof.
15. The product of embodiments 7, 9 or 12-14 wherein the composition comprises an organic acid content (as sum of mono- and di-basic salts) in the range of about 0.10% to about 0.4%, or about 0.18% to about 0.30%, or about 0.20% to about 0.28%, by weight of the total weight of the packaging solution.
16. The product of embodiment 8 wherein the phosphate buffer comprises from about 0.3 wt %) to about 0.9 wt %, from about 0.4 wt % to about 0.85 wt %, from about 0.5 wt % to about 0.8 wt % or from about 0.6 wt % to about 0.75 wt % of at least one phosphate compound by weight of the total weight of the packaging solution.
17. The product of any of the foregoing or succeeding embodiments further comprising at least one tonicity agent.
18. The product of embodiment 17 wherein the tonicity adjusting agents are selected from ionic salts and non-ionic compounds.
19. The product of embodiment 18 wherein the non-ionic compounds may be selected from glycerol, propylene glycol and polyethylene glycols.
20. The product of embodiment 18 wherein the ionic salts may be present in the composition in concentrations to provide osmolalities of at least about 240, about 260 about 280 or about 300 mOsm/kg.
21. The product of embodiment 18 wherein the ionic salts may be present in the composition in concentrations to provide an osmolality from the ionic salts alone of about 240 mOsm/kg to about 450 mOsm/kg, about 260 mOsm/kg to about 430 mOsm/kg, about 280 mOsm/kg to about 420 mOsm/kg or 260 mOsm/kg to about 300 mOsm/kg.
22. The product of any of the preceding or succeeding embodiments further comprising ionic salts and non-ionic compound tonicity agents, wherein the composition has an osmolality of about 240 to about 600, about 260 to about 500, about 280 to about 500, about 300 to about 500 or about 200 to about 430 mOsm/kg.
23. The product of embodiment 18 wherein the non-ionic compound comprises propylene glycol in amounts between about 0.5 to about 3 wt %, 0.5 to about 2 wt % or about 0.5 to about 1.5 wt %, based upon the packaging solution.
24. The product of embodiment 18 wherein the non-ionic compound comprises polyethylene glycol may be included in amounts between 0.5 to about 3 wt %, or about 0.5 to about 2 wt % or about 0.5 to about 1.5 wt % based upon the packaging solution.
25. The product of any of the preceding or succeeding embodiments further comprising at least one therapeutic agent.
26. The product of embodiment 25 wherein the therapeutic agent is hydrolytically unstable at neutral pH.
27. The product of embodiment 25 wherein the therapeutic agent is selected from the atropine, atropine, atropine sulfate, brimonidine, brimonidine tartrate, lifitigrast, perfluorohexyloxtane, ketotifen, ketotifen fumarate, olopatadine, alcaftadine, azelastine, cetirizine, latanoprost, bimatoprost, pilocarpine, hydrocortisone, loteprednol, prednisolone, prednisolone acetate, dexamethasone, ciprofloxacin, amoxicillin, tobramycin, meomycin, bacitracin, polymyxin B, gentamycin and mixtures thereof.
28. The product of embodiment 25 wherein the therapeutic agent comprises atropine, atropine sulfate, and mixtures thereof.
29. The product of embodiment 28 wherein the composition comprises about 0.001 to about 1.5 wt %, about 0.1 to about 1.5 wt %, or between about 0.001 to about 0.05 wt %, or about 0.01 to about 0.03 wt % atropine compound based upon the total weight of the packaging solution.
30. The product of embodiments 25-29 wherein said composition is free of borate compounds.
31. The product of any of the preceding or foregoing embodiments further comprising at least one sequestering agent.
32. The product of embodiment 31 wherein the sequestering agent is selected from disodium ethylene diamine tetraacetate (EDTA), diethylenetriamine pentaacetic acid (DTPA) and ophthalmically compatible salts thereof, alkali metal hexametaphosphate, citric acid, sodium citrate and the like and mixtures thereof.
33. The product of embodiment 31 wherein the sequestering agent comprises EDTA.
34. The product of embodiments 31-33 wherein the sequestering agent is present in amounts from about 0.008 to about 0.075 or about 0.01 to about 0.075 wt %.
35. The product of embodiments 31-33 wherein the sequestering agent is present in amounts between about 0.008 and about 0.012 wt % or about 0.1 wt %, based on the total packaging solution composition.
36. The product of any of the preceding or foregoing embodiments further comprising at least one demulcent.
37. The product of embodiments 2-6 further comprising a therapeutic agent and wherein the buffer is free from borate.
38. The product of any of the preceding or foregoing embodiments wherein the packaging solution is free from preservatives.
39. The product of any of the preceding or foregoing embodiments wherein the sealed ophthalmic product is heat sterilized.
40. The product of any of the preceding or succeeding embodiments wherein the silicone hydrogel comprises at least one hydrolysable silicone monomer unit, the packaging solution comprises at least one therapeutic agent and the buffer is a phosphate buffer.
41. The product of embodiment 40 wherein the therapeutic agent comprises atropine or an ophthalmically compatible salt thereof.
42. The product of embodiment 41 or 42 wherein the silicone hydrogel contact lens is a myopia control contact lens.
43. The product of any of the preceding embodiments further comprising a demulcent polymer.
44. The product of any of the preceding embodiments wherein the demulcent polymer is selected from block copolymers of polyethyleneoxide (PEO) and polypropyleneoxide (PPO), polyvinyl alcohol, polyvinyl pyrrolidone, polyacrylic acid, polyethers, hyaluronic acid and hyaluronic acid derivatives, chitosan, polysorbates, dextrans, cellulosic derivatives, acyclic polyamides, salts thereof and mixtures thereof.
45. The product of any of the preceding or succeeding embodiments further comprising additional components selected from wetting agents, therapeutic agents, sequestering agents, viscosity builders, antioxidants, and the like and mixtures thereof.
46. A sealed ophthalmic product comprising:
   a contact lens package comprising a sealed compartment comprising at least one sterile silicone hydrogel contact lens and a buffered packaging solution having a pH of about 4.5 to 6.5, at least one non-ionic tonicity agent in an amount between about 0.5 wt % and amounts that increase the osmolality of the packaging solution by up to about 150, about 200 or about 280 mOsm/kg.
47. The product of embodiment 25 or 37 wherein the therapeutic agent is selected from antimuscarinic agents, prostaglandins, cholinergic agonists, steroids, antibiotics, anti-inflammatories and dry eye drugs.
48. The product of embodiment 25 or 37 wherein the therapeutic agent is selected from atropine, atropine sulfate, brimonidine, brimonidine tartrate, lifitigrast, perfluorohexyloxtane, ketotifen, ketotifen fumarate, olopatadine, alcaftadine, azelastine, cetirizine, latanoprost, bimatoprost, pilocarpine, prednisolone, prednisolone acetate and dexamethasone.
49. A sealed ophthalmic product comprising:
   a package comprising a sealed compartment comprising at least one sterile ocular insert and a buffered packaging solution having a pH of about 4.5 to 6.5.

What is claimed is:

1. A sealed ophthalmic product or kit, comprising:
a contact lens package comprising a sealed compartment comprising at least one sterile silicone hydrogel contact lens and a buffered packaging solution having a pH of about 4.5 to about 6.

2. The product of claim 1 wherein the silicone hydrogel contact lens comprises units from at least one hydrolysable silicone monomer.

3. The product of claim 2 wherein the hydrolysable silicone monomer is selected from 3-[tris(trimethylsiloxy)silyl]propyl methacrylate (TRIS), 3-methacryloxypropylbis(trimethylsiloxy)methylsilane, N-[3-tris(trimethylsiloxy)silyl]-propyl acrylamide (TRIS-Am), 2-hydroxy-3-[3-methyl-3,3-di(trimethylsiloxy)silylpropoxy]-propyl methacrylate (SiMAA), 2-hydroxy-3-methacryloxypropyloxypropyl-tris(trimethylsiloxy)silane and 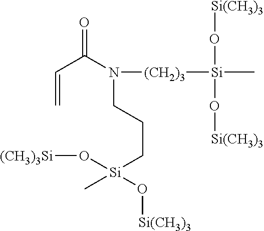
And mixtures thereof.

4. The product of claim 2 wherein the hydrolysable silicone monomer is selected from 3-[tris(trimethylsiloxy)silyl]propyl methacrylate (TRIS), 3-methacryloxypropylbis(trimethylsiloxy)methylsilane, N-[3-tris(trimethylsiloxy)silyl]-propyl acrylamide (TRIS-Am), 2-hydroxy-3-[3-methyl-3,3-di(trimethylsiloxy)silylpropoxy]-propyl methacrylate (SiMAA), 2-hydroxy-3-methacryloxypropyloxypropyl-tris(trimethylsiloxy)silane and mixtures thereof.

5. The product of claim 2 wherein the silicone hydrogel is selected from include galyfilcon, senofilcon, stenfilcon, samfilcon, balafilcon, delefilcon.

6. The product of claim 1 wherein the pH is about 5 to about 6.

7. The product of claim 1 wherein the buffered packaging solution comprises one or more phosphate compound, sulfate compound, borate compound or organic acid buffers.

8. The product of claim 1 wherein the buffered packaging solution is a phosphate buffer system comprising one or more phosphate compound.

9. The product of claim 2 wherein the buffered packaging solution is an organic acid buffer comprising one or more organic acid buffer or a mixture of organic acid compounds and borate or phosphate compounds.

10. The product of claim 1 wherein the buffered packaging solution is a borate buffer system comprising one or more borate compound.

11. The product of claim 10 wherein the composition comprises from about from about 0.2 wt % to less about 1.5 wt % of at least one borate compound, by weight of the packaging solution.

12. The product of claim 9 wherein the organic acid buffer is selected from phytic acid, mellitic acid, maleic acid and salts thereof (such the sodium or potassium salts of the organic acids) and mixtures thereof.

13. The product of claim 9 wherein the organic acid buffer is selected from maleic acid, its sodium or potassium salts and mixtures thereof.

14. The product of claim 12 wherein the composition comprises an organic acid content (as sum of mono- and di-basic salts) in the range of about 0.10% to about 0.4%, by weight of the packaging solution.

15. The product of claim 8 wherein the phosphate buffer comprises from about 0.4 wt % to about 0.85 wt %, of at least one phosphate compound by weight of the the packaging solution.

16. The product of claim 1 wherein the packaging solution further comprising at least one tonicity agent.

17. The product of claim 16 wherein the tonicity adjusting agents are selected from ionic salts and non-ionic compounds.

18. The product of claim 17 wherein the non-ionic compounds may be selected from glycerol, propylene glycol and polyethylene glycols.

19. The product of embodiment 17 wherein the ionic salts may be present in the composition in concentrations to provide an osmolality from the ionic salts alone of about 240 mOsm/kg to about 450 mOsm/kg.

20. The product of claim 17 wherein the packaging solution comprises ionic salts and non-ionic compound tonicity agents, wherein the composition has an osmolality of about 240 to about 600m Osm/kg.

21. The product of claim 18 wherein the non-ionic compound comprises propylene glycol in amounts between about 0.5 to about 3 wt %, based upon the packaging solution.

22. The product of claim 18 wherein the non-ionic compound comprises polyethylene glycol in amounts between 0.5 to about 3 wt %, based upon the packaging solution.

23. The product of claim 2 further comprising at least one therapeutic agent.

24. The product of claim 23 wherein the therapeutic agent is hydrolytically unstable at neutral pH.

25. The product of claim 23 wherein the therapeutic agent comprises at least one atropine compound selected from atropine, atropine sulfate, and mixtures thereof.

26. The product of claim 25 wherein the composition comprises about 0.001 to about 0.05 wt % atropine compound based upon the total weight of the packaging solution.

27. The product of claim 25 wherein said composition is free of borate compounds.

28. The product of claim 1 further comprising at least one sequestering agent.

29. The product of claim 28 wherein the sequestering agent is selected from disodium ethylene diamine tetraacetate (EDTA), diethylenetriamine pentaacetic acid (DTPA) and ophthalmically compatible salts thereof, alkali metal hexametaphosphate, citric acid, sodium citrate and the like and mixtures thereof.

30. The product of claim 28 wherein the sequestering agent comprises EDTA.

31. The product of claim 30 wherein the sequestering agent is present in amounts from about 0.008 to about 0.075 wt %.

32. The product of claim 2 further comprising a therapeutic agent and wherein the buffer is free from borate.

33. The product of claim 1 wherein the packaging solution is free from preservatives.

34. The product of claim 1 wherein the sealed ophthalmic product is heat sterilized.

35. The product of claim 1 wherein the silicone hydrogel comprises at least one hydrolysable silicone monomer unit, the packaging solution comprises at least one therapeutic agent and the buffer is a phosphate buffer.

36. The product of claim 35 wherein the therapeutic agent comprises atropine or an ophthalmically compatible salt thereof.

37. The product of claim 36 wherein the silicone hydrogel contact lens is a myopia control contact lens.

38. A sealed ophthalmic product comprising:
a contact lens package comprising a sealed compartment comprising at least one sterile silicone hydrogel contact lens and a buffered packaging solution having a pH of about 4.5 to 6.5, and osmolality of about 240 to about 600 mOsm/kg, and at least one non-ionic tonicity agent in an amount between about 0.5 wt % and amounts that increase the osmolality of the packaging solution by up to about 280 mOsm/kg.

39. The product of claim 38 further comprising at least one therapeutic agent selected from the group consisting of antimuscarinic agents, prostaglandins, cholinergic agonists, steroids, antibiotics, anti-inflammatories and dry eye drugs.

40. The product of claim 39 wherein the therapeutic agent is selected from atropine, atropine sulfate, brimonidine, brimonidine tartrate, lifitigrast, perfluorohexyloxtane, ketotifen, ketotifen fumarate, olopatadine, alcaftadine, azelastine, cetirizine, latanoprost, bimatoprost, pilocarpine, prednisolone, prednisolone acetate and dexamethasone.

41. A sealed ophthalmic product comprising:
a package comprising a sealed compartment comprising at least one sterile ocular insert and a buffered packaging solution having a pH of about 4.5 to 6.5.
